(19) United States
(12) Patent Application Publication
(10) Pub. No.: US 2011/0076245 A1
(43) Pub. Date: Mar. 31, 2011

(54) NOVEL INVERSE LATICES THAT ARE FREE OF OXYETHYLENE DERIVATIVES, AND COSMETIC, DERMOCOSMETIC, DERMOPHARMACEUTICAL OR PHARMACEUTICAL COMPOSITIONS COMPRISING SAME

(75) Inventors: Olivier Braun, Castres (FR); Georges Da Costa, Saix (FR); Hervé Rolland, Castres (FR); Paul Mallo, Croissy-Sur-Seine (FR)

(73) Assignee: Societe D"exploitation De Produits Pour Les Industries Chimiques Seppic, Paris (FR)

(21) Appl. No.: 12/994,966

(22) PCT Filed: Jun. 22, 2009
(86) PCT No.: PCT/FR2009/051190

§ 371 Date: Nov. 29, 2010

(30) Foreign Application Priority Data
Jun. 27, 2008 (FR) 0854324

(51) Int. Cl. A61K 8/81; A61Q 17/04; A61Q 1/14; A61Q 1/02; A61Q 5/00; A61Q 19/00; A61Q 19/10

(52) U.S. Cl. 424/59; 424/70.16; 514/772.3

(57) ABSTRACT

Novel inverse latices comprising, as inverting agents, at least one compound of formula (I): R₃—[O—CH₂—CH(OH)—CH₂]_{P}—OH (I) in which R₃ represents a linear or branched aliphatic radical comprising from 6 to 18 carbon atoms and p an integer greater than or equal to and less than or equal to 20; and cosmetic, dermocosmetic, dermopharmaceutical or pharmaceutical compositions comprising them.

The present patent application relates to water-in-oil inverse latices, to the method for preparing same and to the use thereof as thickeners and/or emulsifiers for skin, hair and scalp care products and for the production of cosmetic, dermocosmetic, dermopharmaceutical or pharmaceutical preparations.

Synthetic thickening polymers, in the form of inverse latices, are described as being able to be used in the production of topical compositions, in the European patent applications published under the numbers EP 0 716 594, EP 1 047 716, EP 1 056 805 and EP 0 503 853.

However, most of these inverse lattices contain poly(ethylenedioxy) derived surfactants. However, most of these compounds are now considered with mistrust by cosmeticians and some of them sometimes create intolerance reactions on certain sensitive skins.

Attempts have been made to replace this class of surfactant with surfactants derived from sugars, such as those described in the European patent applications published under numbers EP 1 055 707 and EP 1 055 451. Nevertheless, their use makes it possible to obtain only imperfect stable dispersions when the inverse lattices invert in water.

For this reason, the inventors have focused on the search for new polymer emulsions which are better tolerated by the skin while at the same time inverting satisfactorily.

The subject of the invention is a composition in the form of an inverse latex comprising, for 100% of its mass:
a) from 10% by mass to 80% by mass of a linear, branched or crosslinked polymer (P) of 2-methyl-2-[(1-oxo-2-propenyl)amino]-1-propanesulfonic acid (also known as 2-acrylamido-2-methylpropanesulfonic acid) which is partially or totally salified, and of optionally one or more monomers chosen from acrylic acid, methacrylic acid, itaconic acid, maleic acid or 3-methyl-3-[(1-oxo-2-propenyl)amino]butanoic acid, the carboxylic function of said monomers being in acid form, partially salified or totally salified, acrylamide, methacrylamide, diacetoneacrylamide, N,N-dimethylacrylamide, N-isopropylacrylamide, N-[2-hydroxy-1,1-bis(hydroxymethyl)-ethyl]propenamide [or tris(hydroxymethyl)acrylamidomethane or N-tris(hydroxymethyl)methylacrylamide also known as THAM], (2-hydroxyethyl)acrylate, (2,3-dihydroxypropyl)acrylate, (2-hydroxyethyl)methacrylate, (2,3-dihydroxypropyl)methacrylate or vinylpyrrolidone; 2,N,N,N-tetramethyl-2-[(1-oxo-2-propenyl)amino]propanammonium chloride, bromide or iodide, 2,N,N-trimethyl-2-[(1-oxo-2-propenyl)amino]propanammonium chloride, bromide or iodide, N,N,N-trimethyl-2-[(1-oxo-2-propenyl)oxy]ethanammonium chloride, bromide or iodide, N,N,N-trimethyl-3-[(1-oxo-2-propenyl)oxy]propanammonium chloride, bromide or iodide, N,N,N-trimethyl-2-[(1-oxo-2-propenyl)amino]propanammonium chloride, bromide or iodide, or diallyldimethylammonium chloride, bromide or iodide;
b) from 5% by mass to 10% by mass of an emulsifying system (S₁) of water-in-oil (W/O) type,
c) from 1% by mass to 50% by mass of water,
d) from 5% by mass to 50% by mass of oil,
e) up to 5% by mass of an emulsifying system (S₂) of oil-in-water (O/W) type comprising at least one compound of formula (I):

R₃—(C═O)—[O—CH₂—CH(OH)—CH₂]ₚ—OH (I)

in which R₃ represents a linear or branched aliphatic radical containing from 5 to 17 carbon atoms and p an integer greater than or equal to 3 and less than or equal to 20, it being understood that said emulsifying system (S₂) of oil-in-water (O/W) type does not comprise poly(ethylenedioxy) derived surfactant compounds.

In the composition as defined above, the polymer (P) present in the composition which is the subject of the invention may be a homopolymer or a polymer formed from several different types of monomers. It is mainly a homopolymer, a copolymer, a terpolymer or a tetrapolymer.

The term “branched polymer” denotes, for (P), a nonlinear polymer which has pendent chains so as to obtain, when this polymer is dissolved in water, a high state of entanglement leading to very high low-gradient viscosities.

The term “crosslinked polymer” denotes, for (P), a nonlinear polymer which is in the form of a three-dimensional network that is water-insoluble but water-swellable and which thus leads to the production of a chemical gel.

According to one particular aspect of the present invention, in the polymer (P) as defined above, the molar proportion of 2-methyl-2-[(1-oxo-2-propenyl)amino]-1-propanesulfonic acid monomeric unit is greater than or equal to 30%, and more particularly greater than or equal to 40%.

The composition according to the invention may comprise a linear polymer, a crosslinked polymer or a branched polymer.

According to one particular aspect of the present invention, the polymer (P) is crosslinked.

When the polymer (P) is crosslinked, it is more particularly crosslinked with a diethylene or polyethylene compound in a molar proportion, expressed relative to the total molar amount of monomers used, of less than or equal to 0.25% and more particularly less than or equal to 0.05%, and most particularly between 0.005% and 0.01%. Preferably, the crosslinking agent and/or the branching agent is chosen from ethylene glycol dimethacrylate, diethylene glycol diacrylate, ethylene glycol diacrylate, diallylurea, triallylamine, trimethylolpropane triacrylate, methylenebis(acrylamide) or a mixture of these compounds, diallyloxyacetic acid or one of its salts, such as sodium diallyloxyacetate, or a mixture of these compounds.

For the constituent monomers comprising an acid function, of the polymer (P) of the composition as defined above, the term “salified” indicates that it is a question of alkali metal salts, such as sodium or potassium salts, or salts of nitrogenous bases, for instance the ammonium salt, the lysine salt or the monoethanolamine salt (HO—CH₂—CH₂—NH₄⁺).

According to one particular aspect of the present invention, the term “salified” signifies that the acid function is salified in the form of a sodium salt.

In the composition as defined above, the emulsifying system (S₁) of water-in-oil (W/O) type consists either of a sole emulsifying surfactant or of a mixture of emulsifying surfactants, provided that said mixture has an HLB value that is sufficiently low to generate water-in-oil emulsions. As emulsifying surfactants of water-in-oil type, mention may be made, for example, of sorbitan esters, such as sorbitan oleate, for instance the product sold by the company SEPPIC under the name Montane™ 80, sorbitan isostearate, for instance the product sold by the company SEPPIC under the name Montane™ 70, or sorbitan sesquioleate, for instance the product sold by the company SEPPIC under the name Montane™ 83. Mention may also be made of polyesters with a molecular weight of between 1000 and 3000, products resulting from condensation between a poly(isobutenyl)succinic acid or the anhydride thereof, such as Hypermer™ 2296 sold by the company Uniqema or else polyglycerol polyhydroxystearate (Dehymuls™ PGPH sold by the company Cognis) or, finally, block copolymers with a molecular weight of between 2500 and 3500, such as Hypermer™ B246 sold by the company Uniqema or Simaline™ IE 200 sold by the company SEPPIC.

In the composition as defined above, the emulsifying system (S₂) of oil-in-water (O/W) type used in the particular aspect of the composition as defined above is composed either of a single emulsifying surfactant or of a mixture of emulsifying surfactants, with the condition that said mixture has an HLB value that is sufficiently higher to give oil-in-water emulsions.

According to one particular aspect of the present invention, the emulsifying system (S₂) of oil-in-water (O/W) type is essentially composed of one or more compounds of formula (Ia) corresponding to formula (I) as defined above, in which R₃ represents a linear or branched aliphatic radical containing from 11 to 17 carbon atoms.

According to one particular aspect of the present invention, the emulsifying system (S₂) of oil-in-water (O/W) type is essentially composed of one or more compounds of formula (Ib) corresponding to formula (I) as defined above, in which p is equal to 10.

As examples of commercial products composed of one or more of said compounds of formulae (I), (Ia) and/or (Ib), mention may be made, for example, of:

Polyado™ 10-1-O KFG sold by the company Lonza, consisting essentially of decaglyceryl monooleate;

Nikkol™ Decaglyn™ 1-IS sold by the company Nikko Chemicals, consisting essentially of decaglyceryl monoisostearate;

Nikkol™ Decaglyn™ 1-L sold by the company Nikko Chemicals, consisting essentially of decaglyceryl monolaurate;

Nikkol™ Decaglyn™ 1-LN sold by the company Nikko Chemicals, consisting essentially of decaglyceryl monolinoleate;

Nikkol™ Decaglyn™ 1-M sold by the company Nikko Chemicals, consisting essentially of decaglyceryl monomyristate;

Drewpol™ 10-1 CCK sold by the company Stepan, consisting essentially of decaglyceryl monocaprylate.

According to one particular aspect of the present invention, the inverse latex also comprises up to 30% by mass of co-surfactants such as, for example, sorbitan laurate.

According to another particular aspect, the composition as defined above comprises from 20% by mass to 70% by mass and more particularly from 30% by mass to 50% by mass of said polymer P.

In the composition which is the subject of the present invention, the oil is either a commercial mineral oil containing saturated hydrocarbons such as paraffins, isoparaffins or cycloparaffins, having, at ambient temperature, a density between 0.7 and 0.9 and a boiling point above approximately 250° C., for instance Marcol™ 52 sold by Exxon Chemical, or a plant oil such as squalane of plant origin, or a synthetic oil, such as hydrogenated polyisobutene or hydrogenated polydecene, or a fatty alcohol ether of formula (II):

R₁—O═R₂ (II)

in which R₁ and R₂ represent, independently of one another, a linear or branched alkyl radical containing from 5 to 18 carbon atoms, or a mixture of several of these oils with one another or with one or more of said fatty alcohol ethers of formula (II). Marcol™ 52 is a commercial oil which corresponds to the definition of liquid petroleum jellies of the French Codex. It is a white mineral oil in accordance with FDA regulations 21 CFR 172.878 and CFR 178.3620 (a), and it is listed in the United States Pharmacopoeia US XXIII (1995) and in the European Pharmacopoeia (1993).

According to another particular aspect of the composition as defined above, the oil is a compound of formula (II) as defined above, in which R₁ and R₂ represent, independently of one another, an alkyl radical chosen from pentyl, hexyl, 1,3-dimethylbutyl, heptyl, octyl, nonyl, isononyl, decyl, undecyl, dodecyl, tetradecyl, hexadecyl or octadecyl radicals.

According to a more particular aspect of the composition as defined above, the oil is chosen from the following compounds of formula (II):

dioctyl ether, didecyl ether, didodecyl ether, dodecyl octyl ether, dihexadecyl ether, (1,3-dimethylbutyl)tetradecyl ether, (1,3-dimethylbutyl)hexadecyl ether, bis(1,3-dimethylbutyl)ether and dihexyl ether.

The constitutive ethers of formula (II) of the oil phase of the inverse latex which is a subject of the present invention are commercially available or can be prepared by methods known to those skilled in the art, for instance the method described in the European patent published under number EP 0 753 500 B1.

According to another particular aspect of the present invention, the polymer (P) included in the inverse latex as defined above is chosen from:
a homopolymer of 2-methyl-2-[(1-oxo-2-propenyl)amino]-1-propanesulfonic acid partially or totally salified in the form of a sodium salt or of an ammonium salt;
a copolymer of 2-methyl-2-[(1-oxo-2-propenyl)amino]-1-propanesulfonic acid, partially or totally salified in the form of a sodium salt or an ammonium salt, and of (2-hydroxyethyl)acrylate;
a copolymer of 2-methyl-2-[(1-oxo-2-propenyl)amino]-1-propanesulfonic acid and of acrylic acid partially or totally salified in the form of a sodium salt or of an ammonium salt;
a copolymer of 2-methyl-2-[(1-oxo-2-propenyl)amino]-1-propanesulfonic acid partially or totally salified in the form of a sodium salt or of an ammonium salt, and of acrylamide;
a copolymer of 2-methyl-2-[(1-oxo-2-propenyl)amino]-1-propanesulfonic acid partially or totally salified in the form of a sodium salt or of an ammonium salt, and of N,N-dimethylacrylamide;
a terpolymer of 2-methyl-2-[(1-oxo-2-propenyl)amino]-1-propanesulfonic acid partially or totally salified in the form of a sodium salt or of an ammonium salt, of acrylamide and of vinylpyrrolidone;
a terpolymer of 2-methyl-2-[(1-oxo-2-propenyl)amino]-1-propanesulfonic acid partially or totally salified in the form of a sodium salt or of an ammonium salt, of acrylic acid partially or totally salified in the form of a sodium salt or of an ammonium salt, and of acrylamide;
a terpolymer of 2-methyl-2-[(1-oxo-2-propenyl)amino]-1-propanesulfonic acid partially or totally salified in the form of a sodium salt or of an ammonium salt, of acrylic acid partially or totally salified in the form of a sodium salt or of an ammonium salt, and of N,N-dimethylacrylamide;
a tetrapolymer of 2-methyl-2-[(1-oxo-2-propenyl)amino]-1-propanesulfonic acid partially or totally salified in the form of a sodium salt or of an ammonium salt, of (2-hydroxyethyl)acrylate, of acrylic acid partially or totally salified in the form of a sodium salt or of an ammonium salt, and of N-[2-hydroxy-1,1-bis(hydroxymethyl)ethyl]propenamide.

According to another, more particular aspect, the polymer (P) is chosen from:
a crosslinked copolymer of 2-methyl-2-[(1-oxo-2-propenyl)amino]-1-propanesulfonic acid (a) partially or totally salified in the form of a sodium salt, and of (2-hydroxyethyl)acrylate (b), in an (a)/(b) molar ratio between 30/90 and 90/10, and more particularly between 40/60 and 90/10;
a crosslinked copolymer of 2-methyl-2-[(1-oxo-2-propenyl)amino]-1-propanesulfonic acid (a) partially or totally salified in the form of a sodium salt, and of acrylic acid (c) partially or totally salified in the form of a sodium salt, in an (a)/(c) molar ratio of greater than 30/70 and 90/10, and more particularly between 40/60 and 90/10, and most particularly greater than 55/45;
a crosslinked copolymer of 2-methyl-2-[(1-oxo-2-propenyl)amino]-1-propanesulfonic acid (a) partially or totally salified in the form of a sodium salt, and of acrylamide (d), in an (a)/(d) molar ratio of greater than 30/70 and 90/10, and more particularly between 40/60 and 90/10, and most particularly greater than or equal to 50/50;
a crosslinked copolymer of 2-methyl-2-[(1-oxo-2-propenyl)amino]-1-propanesulfonic acid (a) partially or totally salified in the form of a sodium salt, and of N,N-dimethylacrylamide (e), in an (a)/(e) molar ratio of greater than 30/70 and 90/10, and more particularly between 40/60 and 90/10, and most particularly greater than or equal to 50/50;
a crosslinked terpolymer of 2-methyl-2-[(1-oxo-2-propenyl)amino]-1-propanesulfonic acid (a) partially or totally salified in the form of a sodium salt, of acrylamide (d) and of vinylpyrrolidone (f), in an (a)/[(d)+(f)] molar ratio of greater than 30/70 and 90/10, and more particularly between 40/60 and 90/10, and most particularly greater than or equal to 50/50;
a crosslinked terpolymer of 2-methyl-2-[(1-oxo-2-propenyl)amino]-1-propanesulfonic acid (a) partially or totally salified in the form of a sodium salt, of acrylic acid (c) partially or totally salified in the form of a sodium salt, and of acrylamide (d), in an (a)/[(c)+(d)] molar ratio of greater than 30/70 and 90/10 and more particularly between 40/60 and 90/10, and most particularly greater than 55/45;
a crosslinked terpolymer of 2-methyl-2-[(1-oxo-2-propenyl)amino]-1-propanesulfonic acid (a) partially or totally salified in the form of a sodium salt, of acrylic acid (c) partially or totally salified in the form of a sodium salt, and of N,N-dimethylacrylamide (e), in an (a)/(e) molar ratio of greater than 30/70 and 90/10, and more particularly between 40/60 and 90/10, and most particularly greater than or equal to 50/50;
a crosslinked tetrapolymer of 2-methyl-2-[(1-oxo-2-propenyl)amino]-1-propanesulfonic acid (a) partially or totally salified in the form of a sodium salt, of (2-hydroxyethyl)acrylate (b), of acrylic acid (c) partially or totally salified in the form of a sodium salt, and of N-[2-hydroxy-1,1-bis(hydroxymethyl)ethyl]propenamide (g), in an (a)/[(b)+(c)+(g)] molar ratio of greater than 30/70 and less than or equal to 90/10, and more particularly between 40/60 and 90/10, and most particularly greater than or equal to 50/50.

The inverse latex as defined above generally contains from 4% to 10% by weight of emulsifiers. Generally, from 20% to 50%, and more particularly from 25 to 40%, of the total weight of the emulsifiers is of the water-in-oil type and from 80% to 50%, and more particularly from 75% to 60%, is of the oil-in-water type.

The inverse latex as defined above generally contains from 5% to 50%, and preferably from 20% to 25%, of compound of formula (I) as defined above. This latex can also contain one or more additives chosen, in particular, from complexing agents, transfer agents or chain limiters.

The inverse latex as defined above generally contains from 1% by mass to 40% by mass of water.

The inverse latex which is the subject of the present invention is generally prepared by inverse emulsion polymerization, a method known to those skilled in the art.

If necessary or if desired, the water-in-oil emulsion of polymer obtained at the end of the polymerization step can be concentrated in order to remove the desired amount of water, and said emulsifying system (S₂) of oil-in-water (O/W) type and/or said co-surfactant can be added to said emulsion.

The subject of the invention is also the use of the composition as defined above, for preparing a cosmetic, dermopharmaceutical or pharmaceutical topical composition.

A topical composition according to the invention, intended to be applied to the skin, to the hair, to the scalp or to the mucous membranes of humans or animals, can consist of a topical emulsion comprising at least one aqueous phase and at least one oily phase. This topical emulsion may be of the oil-in-water type. More particularly, this topical emulsion can consist of a fluid emulsion, such as a milk or a fluid gel. The oily phase of the topical emulsion can consist of a mixture of one or more oils.

A topical composition according to the invention may be intended for a cosmetic use or be used for preparing a medicament for use in the treatment of skin, scalp and mucous membrane diseases. In the latter case, the topical composition then comprises an active ingredient which can, for example, consist of an anti-inflammatory agent, a muscle relaxant, an antifungal agent or an antibacterial agent.

When the topical composition is used as a cosmetic composition intended to be applied to the skin, the scalp or the mucous membranes, it may optionally comprise an active ingredient, for example a moisturizer, a tanning agent, a sunscreen, an anti-wrinkle agent, a slimming agent, a free-radical scavenger, an anti-acne agent or an antifungal agent.

A topical composition according to the invention usually comprises, for 100% of its total mass, between 0.1% and 10% by mass of the thickener defined above. The pH of the topical composition is preferably greater than or equal to 5.

The topical composition may also comprise compounds conventionally included in compositions of this type, for example fragrances, preservatives, antioxidants, dyes, emollients or surfactants.

According to yet another aspect, the invention relates to the use of the novel thickener in accordance with the invention, mentioned above, for thickening and emulsifying a topical composition comprising at least one aqueous phase.

The composition according to the invention is an advantageous substitute for those sold under the names Sepigel™ 305, Sepigel™ 501, Simulgel™ EG, Simulgel™ NS, Simulgel™ INS 100, Simulgel™ SMS 88, Simulgel™ S, Sepiplus 400, Sepiplus 265, Sepiplus S or Simulgel™ 600 by the applicant, since it also exhibits good compatibility with the other excipients used for the preparation of formulations such as milks, lotions, creams, soaps, bubblebath, balms, shampoos or conditioners. It can also be used with said Sepigel or Simulgel products.

It is in particular compatible with the concentrates described and claimed in international publications WO 92/06778, WO 95/04592, WO 95/13863, WO 96/37285, WO 98/22207 and WO 98/47610 or in FR 2734 496, and with the surfactants described in WO 93/08204.

It is particularly compatible with Montanov™ 68, Montanov™ 82, Montanov™ 202, Montanov™ 14, Montanov™ L or Montanov™ S. It can also be used in emulsions of the type described and claimed in EP 0 629 396 and in the cosmetically or physiologically acceptable aqueous dispersions with an organopolysiloxane compound chosen, for example, from those described in WO 93/05762 or in WO 93/21316.

It can also be used to form cosmetically or physiologically acceptable acid-pH aqueous gels, such as those described in WO 93/07856; it can also be used in combination with nonionic celluloses, in order to form, for example, styling gels, such as those described in EP 0 684 024, or else in combination with fatty acid esters of a sugar, in order to form compositions for treating the hair or the skin, such as those described in EP 0 603 019, or alternatively in shampoos or conditioners as described and claimed in WO 92/21316, or, finally, in combination with an anionic homopolymer such as Carbopol™ in order to form hair treatment products, such as those described in DE 195 23596, or in combination with other thickening polymers.

The composition according to the invention is also compatible with active ingredients such as, for example, self-tanning agents, for instance dihydroxyacetone (DHA) and/or erythrulose, or anti-acne agents; it can therefore be introduced into self-tanning compositions such as those claimed in EP 0 715 845, EP 0 604 249, EP 0 576 188 or in WO 93/07902.

It is also compatible with N-acylated derivatives of amino acids, which allows it to be used in soothing compositions, in particular for sensitive skin, such as those described or claimed in WO 92/21318 or WO 94/27561 or in WO 98/09611.

When the composition as defined above is intended for hair treatment, it comprises more particularly an inverse latex of cationic polymer which is the subject of the present invention.

When the composition as defined above is intended for treatment of the skin and/or the mucous membranes, it comprises more particularly an inverse latex of anionic polymer which is the subject of the present invention.

The inverse latices which are the subject of the present invention can be used as a thickener for textile printing pastes.

The purpose of the examples which follow is to illustrate the present invention.

### A) EXAMPLES OF PREPARATION OF A COMPOSITION ACCORDING TO THE INVENTION

### Example 1

### Preparation of an Inverse Latex of (AMPS, Na Salt)/HEA (90/10) Copolymer, Crosslinked with Methylenebis(acrylamide), in Dioctyl Ether (Composition 1)

a)—The following are introduced into a beaker, with stirring:
632.5 g of a commercial 55% sodium 2-methyl-2-[(1-oxo-2-propenyl)amino]-1-propanesulfonate (AMPS, Na salt) solution,
19.6 g of (2-hydroxyethyl)acrylate (HEA),
0.45 g of sodium diethylenetriaminepentaacetate,
0.105 g of methylenebis(acrylamide).
The pH of this aqueous solution is equal to 4.

b)—An organic phase is prepared by mixing:
240 g of dioctyl ether of plant origin,
16.1 g of sorbitan isostearate (Montane™ 70),
13.4 g of sorbitan monolaurate (Montane™ 20), and
2.9 g of polyglyceryl polyhydroxystearate (Dehymul™ PGPH).

c)—The aqueous phase is gradually introduced into the organic phase and the whole is stirred vigorously by means of an Ultra-Turrax™ mixer sold by IKA. The emulsion obtained is then transferred into a polymerization reactor, subjected to sparging with nitrogen and then cooled to approximately 5-6° C. 2.5 ml of a solution containing 0.64% by weight of cumene hydroperoxide in dioctyl ether, and 0.134 g of sodium persulfate in 5 g of water are then added, followed, after homogenization of the solution, by an aqueous solution of sodium metabisulfite (0.8% in water) for approximately 60 minutes at a rate of approximately 0.15 ml/minute, while allowing the temperature to rise to the temperature at the end of polymerization. The reaction medium is then kept at this temperature for approximately 90 minutes. 3% of decaglyceryl monolaurate (Decaglyn™ 1L) are then added, and the desired water-in-oil emulsion is obtained.

Evaluation of the Properties

Viscosity in water at 3% of the latex (Brookfield RVT spindle 6, speed 5):

η=112 000 mPa·s.

Viscosity at 3% of the latex in saline (0.1% NaCl) (Brookfield RVT spindle 3, speed 5): η=9580 mPa·s.

Viscosity of the latex at 25° C. (Brookfield RVT spindle 3, speed 20):

η=1800 mPa·s.

### Example 2

### Preparation of an Inverse Latex of (AMPS, Na Salt)/HEA/THAM/AA (84/10/4/2) Tetrapolymer, Crosslinked with Methylenebis(acrylamide), in Dioctyl Ether (Composition 2)

a) The following are introduced into a beaker, with stirring:
627.6 g of a commercial 55% sodium 2-methyl-2-[(1-oxo-2-propenyl)amino]-1-propanesulfonate (AMPS, Na salt) solution,
20.8 g of (2-hydroxyethyl)acrylate (HEA),
0.45 g of sodium diethylenetriaminepentaacetate (Versenex™ 80),
12.6 g of tris(hydroxymethyl)acrylamidomethane (THAM),
2.6 g of acrylic acid,
0.028 g of methylenebis(acrylamide).
The pH of this aqueous solution is equal to 4.

b)—An organic phase is prepared by mixing:
160 g of dioctyl ether of plant origin (Cosmacol™ OE),
90 g of Isopar™ H,
15 g of sorbitan isostearate (Montane™ 70),
10 g of Hypermer™ 6212, and
7.4 g of Blemmer™ PLE 200.

c)—The aqueous phase is gradually introduced into the organic phase and the whole is stirred vigorously by means of an Ultra-Turrax™ mixer sold by IKA. The emulsion obtained is then transferred into a polymerization reactor, subjected to sparging with nitrogen and then cooled to approximately 5-6° C. 2.5 ml of a solution containing 0.64% by weight of cumene hydroperoxide in dioctyl ether, and 0.134 g of sodium persulfate in 5 g of water are then added, followed, after homogenization of the solution, by an aqueous solution of sodium metabisulfite (0.8% in water) for approximately 60 minutes at a rate of approximately 0.15 ml/minute, while allowing the temperature to rise to the temperature at the end of polymerization. The reaction medium is then kept at this temperature for approximately 90 minutes. 5% of decaglyceryl monolaurate (Decaglyn™ 1L) are then added, and the desired water-in-oil emulsion is obtained.

Evaluation of the Properties

Viscosity of the self-invertible inverse latex at 25° C. (Brookfield RVT spindle 5, speed 20):

η=19 800 mPa·s.

Viscosity in water at 2% of the self-invertible inverse latex (Brookfield RVT spindle 6, speed 5):

η=61 600 mPa·s.

Viscosity in water (containing 0.1% sodium chloride) at 2% of the self-invertible inverse latex (Brookfield RVT spindle 4, speed 5):

η=26 900 mPa·s

pH at 2%: 5.5.

### B) EXAMPLES OF FORMULATIONS PREPARED WITH THE COMPOSITIONS ACCORDING TO THE INVENTION

### Example 1

### Aftershave Balm

Formula

| | | |
|---|---|---|
| A | Composition 1: | 1.5% |
| | Water: | q.s. 100% |
| B | Micropearl ™ M100: | 5.0% |
| | Sepicide ™ CI: | 0.50% |
| | Fragrance: | 0.20% |
| | 95° ethanol: | 10.0% |

Procedure

Add B to A.

### Example 2

### Soothing Aftersun Care

Formula

| Mixture of lauryl amino acids: | 0.1% to 5% |
|---|---|
| Magnesium potassium aspartate: | 0.002% to 0.5% |
| Lanol ™ 99: | 10.0% |
| Water: | q.s. 100% |
| Composition 1: | 2.50% |
| Sepicide ™ HB: | 0.3% |
| Sepicide ™ CI: | 0.2% |
| Fragrance: | 0.4% |
| Dye: | 0.03% |

### Example 3

### Moisturizing and Matting Foundation

Formula

| | | |
|---|---|---|
| A | Water: | 20.0% |
| | Butylene glycol: | 4.0% |
| | PEG-400: | 4.0% |
| | Pecosil ™ PS100: | 1.0% |
| | Sodium hydroxide: | q.s. pH = 9 |
| | Titanium dioxide: | 7.0% |
| | Talc: | 2.0% |
| | Yellow iron oxide: | 0.8% |
| | Red iron oxide: | 0.3% |
| | Black iron oxide: | 0.05% |
| B | Lanol ™ 99: | 8% |
| | Caprylic capric triglyceride: | 8% |
| | Montanov ™ 202: | 5.00% |
| C | Water: | q.s. 100% |
| | Micropearl ™ M305: | 2.0% |
| | Tetrasodium EDTA: | 0.05% |
| D | Dow Corning ™ 345: | 4.0% |
| | Ketrol ™ T: | 0.2% |
| | Composition 1: | 0.8% |
| E | Sepicide ™ HB: | 0.5% |
| | Sepicide CI: | 0.3% |
| | Fragrance: | 0.2% |

Procedure

Prepare the mixtures B+D and A+C, at 80° C., and then mix and emulsify the whole.

### Example 4

### Radiance Gel

Formula

| | | |
|---|---|---|
| A | Composition 1: | 4% |
| | Water: | 30% |
| B | Elastine HPM: | 5.0% |
| C | Micropearl ™ M100: | 3% |
| | Water: | 5% |
| D | Sepicide ™ CI: | 0.2% |
| | Sepicide ™ HB: | 0.3% |
| | Fragrance: | 0.06% |
| | 50% sodium pyrolidinonecarboxylate: | 1% |
| | Water: | q.s. 100% |

Procedure

Prepare A; add B, then C, then D.

### Example 5

### Body Milk

Formula

| | |
|---|---|
| Montanov ™ S: | 3.5% |
| Lanol ™ 37T: | 8.0% |
| Solagum ™ L: | 0.05% |
| Water: | q.s.100% |
| Eusolex ™ 4360: | 2.0% |
| Dimethicone 350cPs: | 0.05% |
| Composition 2: | 0.8% |
| Preservative: | 0.2% |
| Fragrance: | 0.4% |

### Example 6

### Makeup-Removing Emulsion with Sweet Almond Oil

Formula

| | |
|---|---|
| Montanov ™ 68: | 5% |
| Sweet almond oil: | 5% |
| Water: | q.s.100% |
| Composition 1: | 0.3% |
| Glycerol: | 5% |
| Preservative: | 0.2% |
| Fragrance: | 0.3% |

### Example 7

### Moisturizing Cream for Greasy Skin

Formula

| | |
|---|---|
| Montanov ™ 68: | 5% |
| Cetylstearyl octanoate: | 8% |
| Octyl palmitate: | 2% |
| Water: | q.s. 100% |
| Composition 1: | 0.6% |
| Micropearl ™ M100: | 3.0% |
| Mucopolysaccharides: | 5% |
| Sepicide ™ HB: | 0.8% |
| Fragrance: | 0.3% |

### Example 8

### Cream with AHA for Sensitive Skin

| Mixture of lauryl amino acids: | 0.1% to 5% |
|---|---|
| Magnesium potassium aspartate: | 0.002% to 0.5% |
| Lanol ™ 99: | 2% |
| Montanov ™ 68: | 5.0% |
| Water: | q.s. 100% |
| Composition 2: | 1.50% |
| Gluconic acid: | 1.50% |
| Triethanolamine: | 0.9% |
| Sepicide ™ HB: | 0.3% |
| Sepicide ™ CI: | 0.2% |
| Fragrance: | 0.4% |

### Example 9

### Soothing Aftersun Care

| Mixture of lauryl amino acids: | 0.1% to 5% |
|---|---|
| Magnesium potassium aspartate: | 0.002% to 0.5% |
| Lanol ™ 99: | 10.0% |
| Water: | q.s. 100% |
| Composition 2: | 2.50% |
| Sepicide ™ HB: | 0.3% |
| Sepicide ™ CI: | 0.2% |
| Fragrance: | 0.4% |
| Dye: | 0.03% |

### Example 10

### Makeup-Removing Milk

| | |
|---|---|
| Montanov ™ S: | 3% |
| Primol ™ 352: | 8.0% |
| Sweet almond oil: | 2% |
| Water: | q.s. 100% |
| Composition 5: | 0.8% |
| Preservative: | 0.2% |

### Example 11

### Fluid Emulsion at Alkaline pH

| | |
|---|---|
| Marcol ™ 82: | 5.0% |
| NaOH: | 10.0% |
| Water: | q.s. 100% |
| Composition 2: | 1.5% |

### Example 12

### Fluid Foundation

| | |
|---|---|
| Simulsol ™ 165: | 5.0% |
| Lanol ™ 84D: | 8.0% |
| Lanol ™ 99: | 5.0% |
| Water: | q.s. 100% |
| Mineral fillers and pigments: | 10.0% |
| Composition 5: | 1.2% |
| Preservative: | 0.2% |
| Fragrance: | 0.4% |

### Example 13

### Antisun Milk

| | |
|---|---|
| Montanov ™ S : | 3.5% |
| Lanol ™ 37T: | 10.0% |
| Parsol ™ MCX: | 5.0% |
| Eusolex ™ 4360: | 2.0% |
| Water: | q.s. 100% |
| Composition 1: | 1.8% |
| Preservative: | 0.2% |
| Fragrance: | 0.4% |

### Example 14

### Leave-on Care Composition

| | |
|---|---|
| Composition 1: | 1.5% |
| Fragrance: | q.s. |
| Preservative: | q.s. |
| Dow Corning ™ X2 8360: | 5.0% |
| Dow Corning ™ Q2 1401: | 15.0% |
| Water: | q.s. 100% |

### Example 15

### Slimming Gel

| | |
|---|---|
| Composition 6: | 5% |
| Ethanol: | 30% |
| Menthol: | 0.1% |
| Caffeine: | 2.5% |
| Extracts of ruscus: | 2% |
| Extract of ivy: | 2% |
| Sepicide ™ HB: | 1% |
| Water | q.s. 100% |

### Example 16

### Ultra-Natural Tinted Cream Gel

Formula

| | | |
|---|---|---|
| A | Water: | 10.0% |
| | Butylene glycol: | 4.0% |
| | PEG-400: | 4.0% |
| | Pecosil ™ PS100: | 1.5% |
| | NaOH: | q.s. pH = 7 |
| | Titanium dioxide: | 2.0% |
| | Yellow iron oxide: | 0.8% |
| | Red iron oxide: | 0.3% |
| | Black iron oxide: | 0.05% |
| B | Lanol ™ 99: | 4.0% |
| | Caprylic capric triglyceride | 4.0% |
| | Sepifeel ™ One: | 1.0% |
| | Composition 5: | 3.0% |
| C | Water: | q.s. 100% |
| | Micropearl ™ M305: | 2.0% |
| | Tetrasodium EDTA: | 0.05% |
| | Dow Corning ™ 245 Fluid: | 4.0% |
| D | Sepicide ™ HB: | 0.5% |
| | Sepicide ™ CI: | 0.3% |
| | Fragrance: | 0.2% |

Procedure

Prepare the mixture B+C, then add A, then D.

### Example 17

### Care for Greasy Skin

Formula

| | | |
|---|---|---|
| A | Micropearl ™ M310 | 1.0% |
| | Composition 5: | 5.0% |
| | Octyl isononanoate: | 4.0% |
| B | Water: | q.s. 100% |
| C | Sepicontrol ™ A5: | 4.0% |
| | Fragrance: | 0.1% |
| | Sepicide ™ HB: | 0.3% |
| | Sepicide ™ CI: | 0.2% |
| D | Capigel ™ 98: | 0.5% |
| | Water: | 10% |

### Example 18

### Cream with AHA

Formula

| | | |
|---|---|---|
| A | Montanov ™ 68: | 5.0% |
| | Lipacide ™ PVB: | 1.05% |
| | Lanol ™ 99: | 10.0% |
| B | Water: | q.s. 100% |
| | Gluconic acid: | 1.5% |
| | TEA (triethanolamine): | 0.9% |
| C | Composition 2: | 1.5% |
| D | Fragrance: | 0.4% |
| | Sepicide ™ HB: | 0.2% |
| | Sepicide ™ CI: | 0.4% |

### Example 19

### Nongreasy Self-Tanning Product for the Face and Body

Formula

| | | |
|---|---|---|
| A | Lanol ™ 2681: | 3.0% |
| | Composition 1: | 2.5% |
| B | Water: | q.s. 100% |
| | Dihydroxyacetone: | 3.0% |
| C | Fragrance: | 0.2% |
| | Sepicide ™ HB: | 0.8% |
| | NaOH (sodium hydroxide): | q.s. pH = 5 |

### Example 20

### Antisun Milk with Monoi de Tahiti Oil

Formula

| | | |
|---|---|---|
| A | Monoi de Tahiti oil: | 10% |
| | Lipacide ™ PVB: | 0.5% |
| | Composition 7: | 2.2% |
| B | Water: | q.s. 100% |
| C | Fragrance: | 0.1% |
| | Sepicide ™ HB: | 0.3% |
| | Sepicide ™ CI: | 0.1% |
| | Parsol ™ MCX: | 4.0% |

### Example 21

### Facial Antisun Care

Formula

| | | |
|---|---|---|
| A | DC ™ 1501: | 4.0% |
| | Composition 5: | 3.5% |
| B | Water: | q.s. 100% |
| C | Fragrance: | 0.1% |
| | Sepicide ™ HB: | 0.3% |
| | Sepicide ™ CI: | 0.21% |
| | Parsol ™ MCX: | 5.0% |
| | Titanium mica: | 2.0% |
| | Lactic acid: | q.s. pH = 6.5 |

### Example 22

### No-Sun Tanning Emulsion

Formula

| | | |
|---|---|---|
| A | Lanol ™ 99: | 15% |
| | Montanov ™ 68: | 5.0% |
| | Parsol ™ MCX: | 3.0% |
| B | Water: | q.s. 100% |
| | Dihydroxyacetone: | 5.0% |
| | Monosodium phosphate: | 0.2% |
| C | Composition 1: | 0.5% |
| D | Fragrance: | 0.3% |
| | Sepicide ™ HB: | 0.8% |
| | NaOH: | q.s. pH = 5 |

### Example 23

### Care Cream

| | |
|---|---|
| Dow Corning ™ 345: | 10% |
| Composition 8: | 0.8% |
| Montanov ™ 68: | 4.5% |
| Preservative: | 0.65% |
| Lysine: | 0.025% |
| EDTA (disodium salt): | 0.05% |
| Ketrol ™ T: | 0.2% |
| Glycerol: | 3.0% |
| Water: | q.s. 100% |

### Example 24

### Care Cream

| | |
|---|---|
| Dow Corning ™ 345: | 10% |
| Composition 1: | 0.8% |
| Montanov ™ 68: | 4.5% |
| Perfluoropolymethyl isopropyl ether: | 0.5% |
| Preservative: | 0.65% |
| Lysine: | 0.025% |
| EDTA (disodium salt): | 0.05% |
| Pemulen ™ TR: | 0.2% |
| Glycerol: | 3% |
| Water: | q.s. 100% |

### Example 25

### Body Milk

Formula

| | | |
|---|---|---|
| A | Simulsol ™ 165: | 5.0% |
| | Lanol ™ 1688: | 12.0% |
| | Lanol ™ 14M: | 2.0% |
| | Cetyl alcohol: | 0.3% |
| | Schercemol ™ OP: | 3% |
| B | Water: | q.s. 100% |
| C | Composition 2: | 0.35% |
| D | Sepicide ™ CI: | 0.2% |
| | Sepicide ™ HB: | 0.5% |
| | Fragrance: | 0.20% |

Procedure

Emulsify B in A at about 75° C.; add C at about 60° C. and then D at about 30° C.

### Example 26

### Body Milk

Formula

| | | |
|---|---|---|
| A | Montanov ™ S: | 3.0% |
| | Glyceryl triheptonate: | 10.0% |
| B | Water: | q.s. 100% |
| C | Composition 2: | 1.0% |
| D | Fragrance: | q.s. |
| | Preservative: | q.s. |

Procedure

Melt A at about 75° C. Emulsify B in A at 75° C.; add C at about 60° C., then D.

### Example 27

### Alcohol-Free Soothing Aftershave Balm

| Mixture of lauryl amino acids: | 0.1% to 5% |
|---|---|
| Magnesium potassium aspartate: | 0.002% to 0.5% |
| Lanol ™ 99: | 2% |
| Sweet almond oil: | 0.5% |
| Water: | q.s.100% |
| Composition 1: | 3% |
| Sepicide ™ HB: | 0.3% |
| Sepicide ™ CI: | 0.2% |
| Fragrance: | 0.4% |

### Example 28

### Satin Body Emulsion

Formula

| | | |
|---|---|---|
| A | Simulsol ™ 165: | 5.0% |
| | Lanol ™ 1688: | 8.50% |
| | Shea butter: | 2% |
| | Liquid paraffin: | 6.5% |
| | Lanol ™ 14M: | 3% |
| | Lanol ™ S: | 0.6% |
| B | Water: | 66.2% |
| C | Micropearl ™ M100: | 5% |
| D | Composition 5: | 3% |
| E | Sepicide ™ CI: | 0.3% |
| | Sepicide ™ HB: | 0.5% |
| | Monteine ™ CA: | 1% |
| | Fragrance: | 0.20% |
| | Vitamin E acetate: | 0.20% |
| | Sodium pyrolidinonecarboxylate: | 1% (moisturizer) |

Procedure

Add C to B, emulsify B in A at 70° C., then add D at 60° C., and then E at 30° C.

### Example 29

### O/W Cream

Formula

| | | |
|---|---|---|
| A | Simulsol ™ 165: | 5.0% |
| | Lanol ™ 1688: | 20.0% |
| | Lanol ™ P: | 1.0% |
| B | Water: | q.s. 100% |
| C | Composition 2: | 2.50% |
| D | Sepicide ™ CI: | 0.20% |
| | Sepicide ™ HB: | 0.30% |

Procedure

Introduce B into A at about 75° C.; add C at about 60° C., then D at about 45° C.

### Example 30

### Nongreasy Antisun Gel

Formula

| | | |
|---|---|---|
| A | Composition 5: | 3.00% |
| | Water: | 30% |
| B | Sepicide ™ C: | 0.20% |
| | Sepicide ™ HB: | 0.30% |
| | Fragrance: | 0.10% |
| C | Dye: | q.s. |
| | Water: | 30% |
| D | Micropearl ™ M100: | 3.00% |
| | Water: | q.s. 100% |
| E | Silicone oil: | 2.0% |
| | Parsol ™ MCX: | 5.00% |

Procedure

Introduce B into A; add C, then D, then E.

### Example 31

### Antisun Milk

Formula

| | | |
|---|---|---|
| A | Montanov ™ S: | 3.0% |
| | Sesame oil: | 5.0% |
| | Parsol ™ MCX: | 5.0% |
| | λ-carrageenan: | 0.10% |
| B | Water: | q.s. 100% |
| C | Composition 1: | 0.80% |
| D | Fragrance: | q.s. |
| | Preservative: | q.s. |

Procedure

Emulsify B in A at 75° C., then add C at about 60° C., then D at about 30° C., and adjust the pH if necessary.

### Example 32

### Massage Gel

Formula

| | | |
|---|---|---|
| A | Composition 2: | 3.5% |
| | Water: | 20.0% |
| B | Dye: | 2 drops/100 g |
| | Water: | q.s. |
| C | Alcohol: | 10% |
| | Menthol: | 0.10% |
| D | Silicone oil: | 5.0% |

Procedure

Add B to A; then add C to the mixture, then D.

### Example 33

### Massage Care Gel

Formula

| | | |
|---|---|---|
| A | Composition 1: | 3.00% |
| | Water: | 30% |
| B | Sepicide ™ CI: | 0.20% |
| | Sepicide ™ HB: | 0.30% |
| | Fragrance: | 0.05% |
| C | Dye: | q.s. |
| | Water: | q.s. 100% |
| D | Micropearl ™ SQL: | 5.0% |
| | Lanol ™ 1688: | 2% |

Procedure

Prepare A; add B, then C, then D.

### Example 34

### Alcohol-Free Soothing Aftershave Balm

Formula

| | | |
|---|---|---|
| A | Lipacide ™ PVB: | 1.0% |
| | Lanol ™ 99: | 2.0% |
| | Sweet almond oil: | 0.5% |
| B | Composition 1: | 3.5% |
| C | Water: | q.s. 100% |
| D | Fragrance: | 0.4% |
| | Sepicide ™ HB: | 0.4% |
| | Sepicide ™ CI: | 0.2% |

### Example 35

### Refreshing Aftershave Gel

Formula

| | | |
|---|---|---|
| A | Lipacide ™ PVB: | 0.5% |
| | Lanol ™ 99: | 5.0% |
| | Composition 1: | 2.5% |
| B | Water: | q.s. 100% |
| C | Micropearl ™ LM: | 0.5% |
| | Fragrance: | 0.2% |
| | Sepicide ™ HB: | 0.3% |
| | Sepicide ™ CI: | 0.2% |

### Example 36

### Gloss Gel

| | |
|---|---|
| Composition 7: | 1.5% |
| Volatile silicone: | 25% |
| Propylene glycol: | 25% |
| Demineralized water: | 10% |
| Glycerol: | q.s. 100% |

### Example 37

### Slimming Gel

| | |
|---|---|
| Composition 6: | 1.5% |
| Lanol ™ 99: | 2% |
| Caffeine: | 5% |
| Ethanol: | 40% |
| Micropearl ™ LM: | 2% |
| Demineralized water: | q.s. 100% |
| Preservative, fragrance: | q.s. |

### Example 38

### Makeup-Removing Milk

| | |
|---|---|
| Simulsol ™ 165: | 4% |
| Montanov ™ 202: | 1% |
| Triglyceride caprylate-caprate: | 15% |
| Pecosil ™ DCT: | 1% |
| Demineralized water: | q.s. |
| Capigel ™ 98: | 0.5% |
| Composition 2: | 1% |
| Proteol ™ APL: | 2% |
| Sodium hydroxide: | q.s. pH = 7 |

### Example 39

### Restructuring “Rinse-Off” Cream Mask for Stressed and Embrittled Hair

| | |
|---|---|
| Ketrol ™ T: | 0.5% |
| Pecosil ™ SPP50: | 0.75% |
| N-cocoyl amino acids: | 0.70% |
| Butylene glycol: | 3.0% |
| Composition 1: | 3.0% |
| Montanov ™ 82: | 3.0% |
| Jojoba oil: | 1.0% |
| Lanol ™ P: | 6.0% |
| Amonyl ™ DM: | 1.0% |
| Lanol ™ 99: | 5.0% |
| Sepicide ™ HB: | 0.3% |
| Sepicide ™ CI: | 0.2% |
| Fragrance: | 0.2% |
| Water: | q.s. 100% |

### Example 40

### Antisun Cream

| | |
|---|---|
| Simulsol ™ 165: | 3% |
| Montanov ™ 202: | 2% |
| C12-C15 benzoate: | 8% |
| Pecosil ™ PS 100: | 2% |
| Dimethicone: | 2% |
| Dow Corning ™ 345: | 5% |
| Parsol ™ MCX: | 6% |
| Eusolex ™ 4360: | 4% |
| Titanium oxide: | 8% |
| Ketrol ™ T: | 0.2% |
| Butylene glycol: | 5% |
| Demineralized water: | q.s. 100% |
| Composition 1: | 1.5% |
| Preservative, fragrance: | q.s. |

### Example 41

### Care Gel for Combination Skin

| | |
|---|---|
| Composition 1: | 4% |
| Plant squalane: | 5% |
| Dimethicone: | 1.5% |
| Sepicontrol ™ A5: | 4% |
| Ketrol ™ T: | 0.3% |
| Water: | q.s. 100% |
| Preservative, fragrance: | q.s. |

### Example 42

### Hair Lotion

| | |
|---|---|
| Butylene glycol: | 3.0% |
| Composition 2: | 3% |
| Simulsol ™ 1293: | 3.0% |
| Lactic acid: | q.s. pH = 6 |
| Sepicide ™ HB: | 0.2% |
| Sepicide ™ CI: | 0.3% |
| Fragrance: | 0.3% |
| Water | q.s. 100% |

### Example 43

### Protective, Relaxing Shampoo

| | |
|---|---|
| Amonyl ™ 675 SB: | 5.0% |
| 28% sodium lauryl ether sulfate: | 35.0% |
| Composition 1: | 3.0% |
| Sepicide ™ HB: | 0.5% |
| Sepicide ™ CI: | 0.3% |
| Sodium hydroxide: | q.s. pH = 7.2 |
| Fragrance: | 0.3% |
| Dye (FDC blue 1/yellow 5): | q.s. |
| Water: | q.s. 100% |

### Example 44

### “Leave-on” Protective Product; Antistress Haircare

| | |
|---|---|
| Ketrol ™ T: | 0.5% |
| Mixture of cocoyl amino acids: | 3.0% |
| Buylene glycol: | 5.0% |
| DC 1501: | 5.0% |
| Composition 1: | 4.0% |
| Sepicide ™ HB: | 0.5% |
| Sepicide ™ CI: | 0.3% |
| Fragrance: | 0.3% |
| Water: | q.s. 100% |

### Example 45

### Cream with Vitamins

| | |
|---|---|
| Simulsol ™ 165: | 5% |
| Montanov ™ 202: | 1% |
| Caprylic/capric triglycerides: | 20% |
| Vitamin A palmitate: | 0.2% |
| Vitamin E acetate: | 1% |
| Micropearl ™ M 305: | 1.5% |
| Composition 1: | 2% |
| Water: | q.s. 100% |
| Preservative, fragrance: | q.s. |

### Example 46

### Antisun Gel

Formula

| | |
|---|---|
| Composition 1: | 3.00% |
| Sepicide ™ CI: | 0.20% |
| Sepicide ™ HB: | 0.30% |
| Fragrance: | 0.10% |
| Dye: | q.s. |
| Silica: | 3.00% |
| Water: | q.s. 100% |
| Silicone oil: | 2.0% |
| Eusolex ™ 4360: | 5.00% |

### Example 47

### Lip Gloss

| | |
|---|---|
| Composition 1: | 1.50% |
| Schermol ™ TISC: | 15.00% |
| Vistanol ™ NPGC: | 15.00% |
| Candurin ™ Paprika: | 0.50% |
| Montanox ™ 80: | 1.00% |
| Antaron ™ V216: | 0.90% |
| Apricot flavoring: | 0.20% |
| Sepicide ™ HB: | 0.50% |
| C Maltidex ™ H16322: | q.s. 100% |

### Example 48

### “Terre de Soleil” Pressed Powder

| | |
|---|---|
| Composition 1: | 2.00% |
| Lanol ™ 99: | 12.00% |
| Sepiwhite ™ MSH: | 1.00% |
| Talc: | 33.00% |
| Micropearl ™ M310: | 3.00% |
| Yellow iron oxide: | 0.80% |
| Red iron oxide: | 0.30% |
| Black iron oxide: | 0.05% |
| Mica: | q.s. 100% |

### Example 49

### Emulsion for Atopic-Prone Skin

| | |
|---|---|
| Arlacel ™ P135: | 2.00% |
| Composition 1: | 1.00% |
| Lanol ™ 1688: | 14.00% |
| Primol ™ 352: | 8.00% |
| Glycerol: | 5.00% |
| Water: | q.s. 100% |
| Magnesium sulfate: | 0.70% |
| Sepicide ™ HB: | 0.30% |
| Sepicide ™ CI: | 0.20% |
| Micropearl ™ M310: | 5.00% |

### Example 50

### Soothing Antisun Care (Water-in-Silicone)

| | |
|---|---|
| Composition 1: | 2.00% |
| DC5225C: | 20.00% |
| DC345: | 10.00% |
| Sepicalm ™ VG: | 3.00% |
| Titanium dioxide MT100VT: | 5.00% |
| Zinc oxide Z-Cote HP1: | 5.00% |
| Sepicide ™ HB: | 0.30% |
| Fragrance: | 0.05% |
| Sepicide ™ CI: | 0.20% |
| Glycerol: | 5.00% |
| Sodium chloride: | 2.00% |
| Water: | q.s. 100% |

### Example 51

### Multiphase Care

| | |
|---|---|
| Composition 1: | 3.00% |
| C12-15 alkyl benzoate: | 25.00% |
| Aquaxyl ™: | 3.00% |
| Sepitonic ™ M3: | 1.00% |
| Sepicide ™ HB: | 0.50% |
| Sepicide ™ CI: | 0.30% |
| Water: | q.s. 100% |

The definitions of the commercial products used in the examples are the following:

Amonyl™ 675 SB is a cocoamidopropyl hydroxy sultaine, sold by the company SEPPIC.

Antaron™ V216 is a synthetic polymer (PVP/hexadecene copolymer) distributed by the company UNIVAR.

Aquaxyl™ is a moisturizer sold by the company SEPPIC.

Arlacel™ P135 No, it is a product sold by Uniquema (now Croda). The SEPPIC equivalent is Simaline IE 200 SF, which is a PEG-30 dipolyhydroxystearate sold by the company SEPPIC.

C Maltidex™ H16322 is a polyol (maltitol syrup) sold by the company Cerestar.

Candurin Paprika is a mixture of potassium aluminum silicate and of iron oxide.

Capigel™ 98 is an acrylate copolymer-based liquid thickener sold by the company SEPPIC.

Dow Corning™ 245 Fluid is cyclomethicone, sold by the company Dow Corning.

DC 345 is a cyclomethicone sold by the company Dow Corning.

DC 5225C is a mixture of cyclopentasiloxane and dimethicone copolyol sold by the company Dow Corning.

DC1501 is a mixture of cyclopentasiloxane and dimethiconol sold by the company Dow Chemical.

Eusolex™ 4360 is benzophenone-3 sold by the company Merck.

Ketrol™ T is xanthan gum sold by the company Kelco.

Lanol™ 2681 is a mixture of coconut caprylate/caprate sold by the company SEPPIC.

Lanol™ 99 is isononyl isononanoate sold by the company SEPPIC.

Lanol™ 1688 is an emollient ester with a nongreasy effect sold by the company SEPPIC.

Lanol™ 14M and Lanol® S are consistency factors sold by the company SEPPIC.

Lanol™ 37T is glyceryl triheptanoate sold by the company SEPPIC.

Lanol™ 84D is dioctyl malate sold by the company SEPPIC.

Lanol™ P is an additive with a stabilizing effect sold by the company SEPPIC.

Lipacide™ PVB is a palmitoylated wheat protein hydrolysate sold by the company SEPPIC.

Marcol™ 82 is a liquid paraffin sold by the company ESSO.

Micropearl™ M100 is an ultrafine powder with a very soft feel and a matting action, sold by the company Matsumo.

Micropearl™ SQL is a mixture of microparticles containing squalane which is released by the action of massaging; it is sold by the company Matsumo.

Micropearl™ LM is a mixture of squalane, poly(methyl methacrylate) and menthol, sold by the company SEPPIC.

Micropearl™ M 305 is a silky water-dispersible powder based on a crosslinked methyl methacrylate copolymer.

Micropearl™ M310 is an ultrafine powder with a very soft feel and a matting action, sold by the company Matsumoto.

Montanov™ S is a pearlescent agent, sold by the company SEPPIC, based on a mixture of alkyl polyglucosides such as those described in WO 95/13863.

Montanov™ 202 (arachidyl glucoside, arachidyl alcohol+behenyl alcohol) is a self-emulsifying composition such as those described in WO 98/17610, sold by the company SEPPIC.

Montanov™ 82 is an emulsifier based on cetearyl alcohol and cocoylglucoside.

Montanov™ 68 is a self-emulsifying composition based on cetearyl glucoside and cetearyl alcohol, as described in WO 92/06778, sold by the company SEPPIC.

Montanox™ 80 is sorbitan oleate polyethoxylated with 20 mol of ethylene oxide, sold by the company SEPPIC.

Monteine™ CA is a moisturizer sold by the company SEPPIC.

MT100VT is a micronized titanium dioxide that has undergone a surface treatment (aluminum hydroxide/stearic acid), distributed by the company Unipex.

Parsol™ MCX is octyl para-methoxycinnamate, sold by the company Givaudan.

Parsol NOX™ is a sunscreen sold by the company Givaudan.

Pecosil™ PS100 is dimethicone copolyol phosphate sold by the company Phoenix.

Pemulen™ TR is an acrylic polymer sold by Goodrich.

Primol™ 352 is a mineral oil sold by the company Exxon.

Proteol™ APL is a foaming surfactant sold by the company SEPPIC.

Schercemol™ OP is an emollient ester with a nongreasy effect.

Schercemol™ TISC is an ester (triisostearyl citrate) sold by the company Scher.

Sepicalm™ VG is a soothing active agent (sodium palmitoyl proline) sold by the company SEPPIC.

Sepitonic™ M3, which is a mixture of magnesium aspartate, zinc gluconate and copper gluconate, is an energizing active agent sold by the company SEPPIC.

Sepicontrol™ A5 is a mixture of capryloyl glycine, sarcosine and extract of Cinnamonum zylanicum, sold by the company SEPPIC, such as those described in international patent application PCT/FR98/01313 filed on Jun. 23, 1998.

Sepicide™ CI, imidazolidinyl urea, is a preservative sold by the company SEPPIC.

Sepicide™ HB, which is a mixture of phenoxyethanol, methylparaben, ethylparaben, propylparaben and butylparaben, is a preservative sold by the company SEPPIC.

Sepifeel™ One is a mixture of palmitoyl proline, magnesium palmitoyl glutamate and magnesium palmitoyl sarcosinate, such as those described in FR 2787323.

Sepiwhite™ MSH is a depigmenting active agent (undecylenoyl phenylalanine) sold by the company SEPPIC.

Simulsol™ 1293 is hydrogenated and ethoxylated castor oil, with an ethoxylation index equal to 40, sold by the company SEPPIC.

Simulsol™ 165 is self-emulsifiable glyceryl stearate sold by the company SEPPIC.

Solagum™ L is a carrageenan sold by the company SEPPIC.

Vistanol™ NPGC is an ester (neopentyl glycol dicaprate) sold by the company Sewa Kasei.

Z-Cote HP1 is a micronized zinc oxide that has undergone a surface treatment, distributed by Gattefosse.

1. A composition in the form of an inverse latex comprising, for 100% of its mass:
a) from 10% by mass to 80% by mass of a linear, branched or crosslinked polymer (P) of 2-methyl-2-[(1-oxo-2-propenyl)amino]-1-propanesulfonic acid (also known as 2-acrylamido-2-methylpropanesulfonic acid) which is partially or totally salified, and optionally one or more monomers chosen from acrylic acid, methacrylic acid, itaconic acid, maleic acid or 3-methyl-3-[(1-oxo-2-propenyl)amino]butanoic acid, the carboxylic function of said monomers being in acid form, partially salified or totally salified, acrylamide, methacrylamide, diacetone acrylamide, N,N-dimethylacrylamide, N-isopropylacrylamide, N-[2-hydroxy-1,1-bis(hydroxymethyl)ethyl]propenamide [or tris(hydroxymethyl)acrylamidomethane or N-tris(hydroxymethyl)methylacrylamide also known as THAM], (2-hydroxyethyl)acrylate, (2,3-dihydroxypropyl)acrylate, (2-hydroxyethyl)methacrylate, (2,3-dihydroxypropyl)methacrylate or vinylpyrrolidone; 2,N,N,N-tetramethyl-2-[(1-oxo-2-propenyl)amino]propanammonium chloride, bromide or iodide, 2,N,N-trimethyl-2-[(1-oxo-2-propenyl)amino]propanammonium chloride, bromide or iodide, N,N,N-trimethyl-2-[(1-oxo-2-propenyl)oxy]ethanammonium chloride, bromide or iodide, N,N,N-trimethyl-3-[(1-oxo-2-propenyl)oxy]propanammonium chloride, bromide or iodide, N,N,N-trimethyl-2-[(1-oxo-2-propenyl)amino]propanammonium chloride, bromide or iodide, or diallyldimethylammonium chloride, bromide or iodide;
b) from 5% by mass to 10% by mass of an emulsifying system (S₁) of water-in-oil (W/O) type,
c) from 1% by mass to 50% by mass of water,
d) from 5% by mass to 50% by mass of oil,
e) up to 5% by mass of an emulsifying system (S₂) of oil-in-water (O/W) type comprising at least one compound of formula (I):
R₃—(C═O)—[O—CH₂—CH(OH)—CH₂]ₚ—OH (I)
in which R₃ represents a linear or branched aliphatic radical containing from 5 to 17 carbon atoms and p an integer greater than or equal to 3 and less than or equal to 20, it being understood that said emulsifying system (S2) of oil-in-water (O/W) type does not comprise poly(ethylenedioxy) derived surfactant compounds.

2. The composition as defined in claim 1, in which the polymer (P) is crosslinked.

3. The composition as defined in claim 1, in which the emulsifying system (S₂) of oil-in-water (O/W) type is essentially composed of one or more compounds of formula (Ia) corresponding to formula (I) as defined above, in which R₃ represents a linear or branched aliphatic radical containing from 11 to 17 carbon atoms.

4. The composition as defined in claim 1, in which the emulsifying system (S₂) of oil-in-water (O/W) type is essentially composed of one or more compounds of formula (Ib) corresponding to formula (I) as defined above, in which p is equal to 10.

5. The composition as defined in claim 1, characterized in that it comprises from 20% by mass to 70% by mass, and more particularly from 30% by mass to 50% by mass of said polymer (P).

6. The composition as defined in claim 1, in which the oil is chosen from the following compounds of formula (II):
dioctyl ether, didecyl ether, didodecyl ether, dodecyl octyl ether, dihexadecyl ether, (1,3-dimethylbutyl)tetradecyl ether, (1,3-dimethylbutyl)hexadecyl ether, bis(1,3-dimethylbutyl)ether and dihexyl ether.

7. The composition as defined in claim 1, characterized in that the polymer (P) is chosen from:
a homopolymer of 2-methyl-2-[(1-oxo-2-propenyl)amino]-1-propanesulfonic acid partially or totally salified in the form of a sodium salt or of an ammonium salt;
a copolymer of 2-methyl-2-[(1-oxo-2-propenyl)amino]-1-propanesulfonic acid, partially or totally salified in the form of a sodium salt or an ammonium salt, and of (2-hydroxyethyl)acrylate;
a copolymer of 2-methyl-2-[(1-oxo-2-propenyl)amino]-1-propanesulfonic acid and of acrylic acid partially or totally salified in the form of a sodium salt or of an ammonium salt;
a copolymer of 2-methyl-2-[(1-oxo-2-propenyl)amino]-1-propanesulfonic acid partially or totally salified in the form of a sodium salt or of an ammonium salt, and of acrylamide;
a copolymer of 2-methyl-2-[(1-oxo-2-propenyl)amino]-1-propanesulfonic acid partially or totally salified in the form of a sodium salt or of an ammonium salt, and of N,N-dimethylacrylamide;
a terpolymer of 2-methyl-2-[(1-oxo-2-propenyl)amino]-1-propanesulfonic acid partially or totally salified in the form of a sodium salt or of an ammonium salt, of acrylamide and of vinylpyrrolidone;
a terpolymer of 2-methyl-2-[(1-oxo-2-propenyl)amino]-1-propanesulfonic acid partially or totally salified in the form of a sodium salt or of an ammonium salt, of acrylic acid partially or totally salified in the form of a sodium salt or of an ammonium salt, and of acrylamide;
a terpolymer of 2-methyl-2-[(1-oxo-2-propenyl)amino]-1-propanesulfonic acid partially or totally salified in the form of a sodium salt or of an ammonium salt, of acrylic acid partially or totally salified in the form of a sodium salt or of an ammonium salt, and of N,N-dimethylacrylamide;
a tetrapolymer of 2-methyl-2-[(1-oxo-2-propenyl)amino]-1-propanesulfonic acid partially or totally salified in the form of a sodium salt or of an ammonium salt, of (2-hydroxyethyl)acrylate, of acrylic acid partially or totally salified in the form of a sodium salt or of an ammonium salt, and of N-[2-hydroxy-1,1-bis(hydroxymethyl)ethyl]propenamide.

8. The composition as defined in claim 7, characterized in that the polymer (P) is chosen from:
a crosslinked copolymer of 2-methyl-2-[(1-oxo-2-propenyl)amino]-1-propanesulfonic acid (a) partially or totally salified in the form of a sodium salt, and of (2-hydroxyethyl)acrylate (b), in an (a)/(b) molar ratio between 30/90 and 90/10, and more particularly between 40/60 and 90/10;
a crosslinked copolymer of 2-methyl-2-[(1-oxo-2-propenyl)amino]-1-propanesulfonic acid (a) partially or totally salified in the form of a sodium salt, and of acrylic acid (c) partially or totally salified in the form of a sodium salt, in an (a)/(c) molar ratio of greater than 30/70 and 90/10, and more particularly between 40/60 and 90/10, and more particularly greater than 55/45;
a crosslinked copolymer of 2-methyl-2-[(1-oxo-2-propenyl)amino]-1-propanesulfonic acid (a) partially or totally salified in the form of a sodium salt, and of acrylamide (b), in an (a)/(d) molar ratio of greater than 30/70 and 90/10, and more particularly between 40/60 and 90/10, and most particularly greater than or equal to 50/50;
a crosslinked copolymer of 2-methyl-2-[(1-oxo-2-propenyl)amino]-1-propanesulfonic acid (a) partially or totally salified in the form of a sodium salt, and of N,N-dimethylacrylamide (e), in an (a)/(e) molar ratio of greater than 30/70 and 90/10, and more particularly between 40/60 and 90/10, and most particularly greater than or equal to 50/50;
a crosslinked terpolymer of 2-methyl-2-[(1-oxo-2-propenyl)amino]-1-propanesulfonic acid (a) partially or totally salified in the form of a sodium salt, of acrylamide (d) and of vinylpyrrolidone (f), in an (a)/[(d)+(f)] molar ratio of greater than 30/70 and 90/10, and more particularly between 40/60 and 90/10, and most particularly greater than or equal to 50/50;
a crosslinked terpolymer of 2-methyl-2-[(1-oxo-2-propenyl)amino]-1-propanesulfonic acid (a) partially or totally salified in the form of a sodium salt, of acrylic acid (c) partially or totally salified in the form of a sodium salt, and of acrylamide (d), in an (a)/[(c)+(d)] molar ratio of greater than 30/70 and 90/10 and more particularly between 40/60 and 90/10, and most particularly greater than 55/45;
a crosslinked terpolymer of 2-methyl-2-[(1-oxo-2-propenyl)amino]-1-propanesulfonic acid (a) partially or totally salified in the form of a sodium salt, of acrylic acid (c) partially or totally salified in the form of a sodium salt, and of N,N-dimethylacrylamide (e), in an (a)/(e) molar ratio of greater than 30/70 and 90/10, and more particularly between 40/60 and 90/10, and most particularly greater than or equal to 50/50;
a crosslinked tetrapolymer of 2-methyl-2-[(1-oxo-2-propenyl)amino]-1-propanesulfonic acid (a) partially or totally salified in the form of a sodium salt, of (2-hydroxyethyl)acrylate (b), of acrylic acid (c) partially or totally salified in the form of a sodium salt, and of N-[2-hydroxy-1,1-bis(hydroxymethyl)ethyl]propenamide (g), in an (a)/[(b)+(c)+(g)] molar ratio of greater than 30/70 and less than or equal to 90/10, and more particularly between 40/60 and 90/10, and most particularly greater than or equal to 50/50.

9. Method of using the composition as defined in, for preparing a cosmetic, dermopharmaceutical or pharmaceutical topical composition.

10. A topical composition, characterized in that it comprises, as thickener, for 100% of its total mass, between 0.1% and 10% by mass of the composition as defined in claim 1.
